(19) United States
(12) Patent Application Publication
(10) Pub. No.: US 2013/0204302 A1
(43) Pub. Date: Aug. 8, 2013

(54) SPINAL IMPLANT SYSTEM AND METHOD

(71) Applicant: William Alan Rezach, Atoka, TN (US)

(72) Inventor: William Alan Rezach, Atoka, TN (US)

(73) Assignee: Warsaw Orthopedic, Inc., Warsaw, IN (US)

(21) Appl. No.: 13/365,002

(22) Filed: Feb. 2, 2012

(51) Int. Cl. A61B 17/70; A61B 17/88

(52) U.S. Cl. 606/260; 606/279

(57) ABSTRACT

A provisional spinal rod comprises a body. A first arm extends between a first end and a second end connected to the body. The first arm is configured for attachment to a first vertebra. A second arm extends between a first end and a second end connected to the body. The second arm is configured for attachment to a second vertebra disposed in an orientation relative to the first vertebra. The second arm is rotatable about the body and relative to the first arm. A locking mechanism is coupled to the body. An insert is disposed between the locking mechanism and the second arm. The arms are relatively rotatable and selectively lockable with the locking mechanism and the insert in the orientation in a template configuration for an implant rod. Methods of use are disclosed.

### TECHNICAL FIELD

The present disclosure generally relates to medical devices for the treatment of musculoskeletal disorders, and more particularly to a surgical system and method for correction of a spine disorder that employs a provisional implant.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, fur stabilization of a treated section of a spine. This disclosure describes an improvement over these prior art technologies.

### SUMMARY

Accordingly, a surgical system and method for treatment of a spine disorder is provided. In one embodiment, in accordance with the principles of the present disclosure, a provisional spinal rod is provided. The provisional spinal rod comprises a body. A first arm extends between a first end and a second end connected to the body. The first arm is configured for attachment to a first vertebra. A second arm extends between a first end and a second end connected to the body. The second arm is configured for attachment to a second vertebra disposed in an orientation relative to the first vertebra. The second arm is rotatable about the body and relative to the first arm. A locking mechanism is coupled to the body. An insert is disposed between the locking mechanism and the second arm. The arms are relatively rotatable and selectively lockable with the locking mechanism and the insert in the orientation in a template configuration for an implant rod.

In one embodiment, in accordance with the principles of the present disclosure, a spinal implant system is provided. The provisional spinal rod comprises a body forming a cavity having a slot and forming a recess. The body is configured to receive a pin. A first arm is rigidly connected to the body and configured for attachment to a first vertebra. A second arm has an end portion configured to receive the pin therethrough to form a pivot connection with the body for rotation of the second arm relative to the first arm. The second arm includes a surface having a three dimensional structure. The second arm is configured for attachment to a second vertebra disposed in an orientation relative to the first vertebra. A locking mechanism is coupled to the body and includes a set screw threaded into the body. An insert is disposed in the recess of the body. The insert provides an interface between the set screw and the surface of the second arm. The insert is configured to interface with the three dimensional structure such that the set screw applies pressure to the insert against the three dimensional structure to lock the orientation of the second arm relative to the first arm. The arms are relatively rotatable and selectively lockable with the locking mechanism in the orientation in a template configuration. A spinal rod implant has a configuration based on the template configuration of the provisional spinal rod.

In one embodiment, a method for treating a spinal disorder is provided. The method comprises the steps of: providing access to a surgical site adjacent a selected section of a spine; providing at least one provisional spinal rod including a body, a first arm extending between a first end and a second end connected to the body, a second arm extending between a first end and a second end connected to the body, the second arm being rotatable about the body and relative to the first arm, a locking mechanism coupled to the body, and an insert disposed between the locking mechanism and the second arm; connecting the first arm with a first vertebra; connecting the second arm with a second vertebra disposed in an orientation relative to the first vertebra; relatively rotating the arms and selectively locking the locking mechanism with the insert such that the arms are disposed in the orientation in a template configuration; and implanting at least one spinal rod implant with the first vertebra and the second vertebra, the at least one spinal rod implant having a configuration based on the template configuration of the at least one provisional spinal rod.

### BRIEF DESCRIPTION OF THE DRAWINGS

The present disclosure will become more readily apparent from the specific description accompanied by the following drawings, in which:

FIG. 1 is a perspective view of one embodiment of components of a spinal implant system in accordance with the principles of the present disclosure;

FIG. 2 is a perspective view of the components of the system shown in FIG. 1 with parts separated;

FIG. 3 is a cutaway plan view of components of the system shown in FIG. 1; and

FIG. 4 is a side view of one embodiment of a spinal implant system in accordance with the principles of the present disclosure disposed with vertebrae.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in teams of a surgical system and method for treatment of a spine disorder. It is envisioned that the surgical system and method may be employed in applications such as correction of deformities such as kyphosis and scoliosis. For example, the surgical system and method can include a temporary holding rod system with an angular adjustment.

In one embodiment, the system and method include an apparatus for stabilizing a musculoskeletal structure that includes at least one arm pivotally connected to a second arm such that angular adjustment can be made and held therebetween. The angular adjustment can be locked in place by a locking mechanism, which permits the angular adjustment to be selectively fixed or held in an orientation. The arms may be connected to portions of the musculoskeletal structure, and the angular adjustment of the holding rod system is made to accommodate an existing or configured angular difference between portions of the musculoskeletal structure. The system includes a temporary rod that has the ability to lock and maintain an angle between both ends for employment with procedures, such as, for example, pedicle subtraction osteotomy (PSO) and vertebral column resection (VCR). The present disclosure includes systems and methods that permit a minimal size instrument/implant to be loaded and allow a surgeon the ability to manipulate the angle between two ends of a working rod.

It is contemplated that one or all of the components of the surgical system may be disposable, peel-pack, pre-packed sterile devices. One or all of the components of the surgical system may be reusable. The surgical system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, direct lateral, postero-lateral, and/or antero-tateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

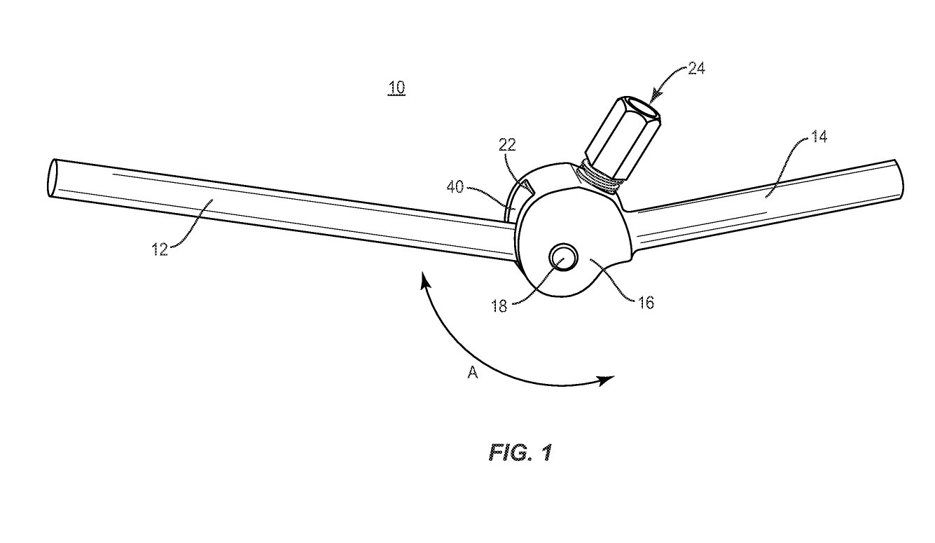

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-3, there are illustrated components of a surgical system, such as, for example, a spinal implant system in accordance with the principles of the present disclosure.

The components of the system can be fabricated from biologically acceptable materials suitable for medical applications, including metals, synthetic polymers, ceramics and bone material and/or their composites, depending on the particular application and/or preference of a medical practitioner. For example, the components of the system, individually or collectively, can be fabricated from materials such as stainless steel alloys, commercially pure titanium, titanium alloys. Grade 5 titanium, super-elastic titanium alloys, cobalt-chronic alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the system may have material composites, including the above materials, to achieve various desired characteristics such as strength, rigidity, elasticity, compliance, biomechanical performance, durability and radiolucency or imaging preference. The components of the system, individually or collectively, may also be fabricated from a heterogeneous material such as a combination of two or more of the above-described materials. The components of the system may be monolithically formed, integrally connected or include fastening elements and/or instruments, as described herein.

The spinal implant system is employed, for example, with an open or mini-open, minimal access and/or minimally invasive including percutaneous surgical technique to provide stabilization of a spine or other musculoskeletal structure, such as, for example, for a surgical treatment to treat various spine pathologies, such as those described herein. The spinal implant system includes a provisional spinal rod 10 including a first arm 14 defining a longitudinal axis and being configured for attachment with vertebrae. A second arm 12 is provided that is pivotally connected to the first arm 14 by a pivot housing or body 16.

Second arm 12 defines a longitudinal axis and is configured for attachment with vertebrae. The first arm 14 and the second area 12 may include a circular cross-section (rod) as depicted or may include other shaped cross-sections, such as, e.g., oval, polygonal, U-shaped, irregular, uniform, non-uniform, variable, tapered, etc. as needed or desired. It is further envisioned that the first arm 14 and the second arm 12 may include a uniform thickness/diameter or may have various other configurations. In addition, the first arm 14 and the second arm 12 may have various surface finishes, such as, for example, rough, threaded for connection with surgical instruments, arcuate, undulating, porous, semi-porous, dimpled, polished and/or textured according to the requirements of a particular application. It is contemplated that the shapes of the arms (12, 14) may be uniformly increasing or decreasing, or have alternate cross-section dimensions along their length. Further, the first arm 14 and the second arm 12 may have pre-defined curvatures (bends) along their length in furtherance of a surgical goal for a particular application.

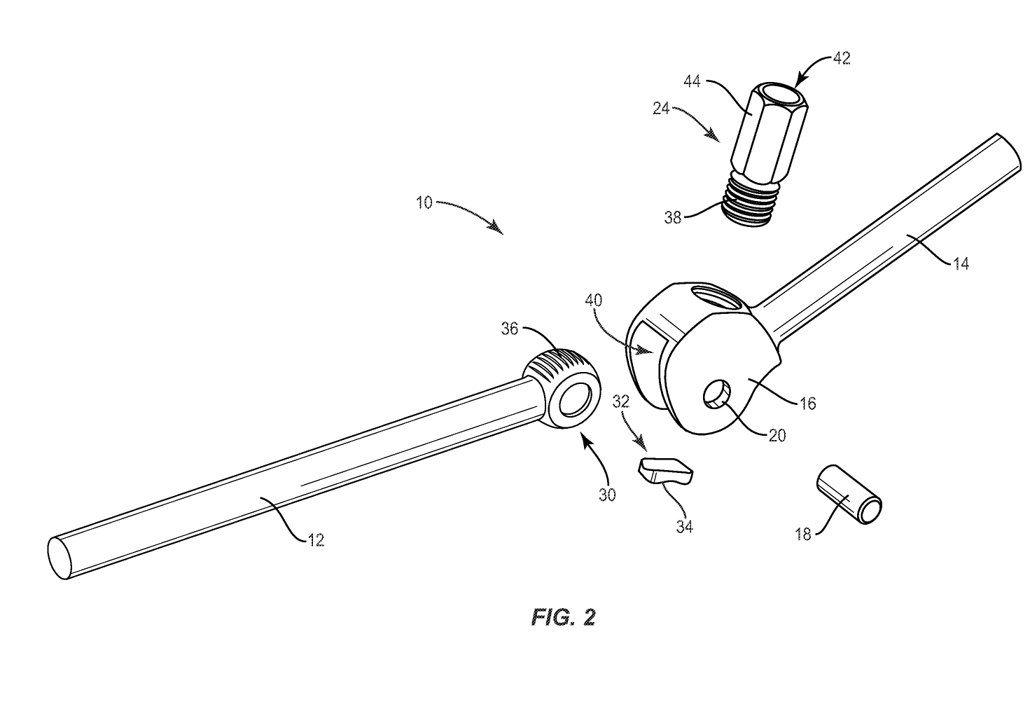

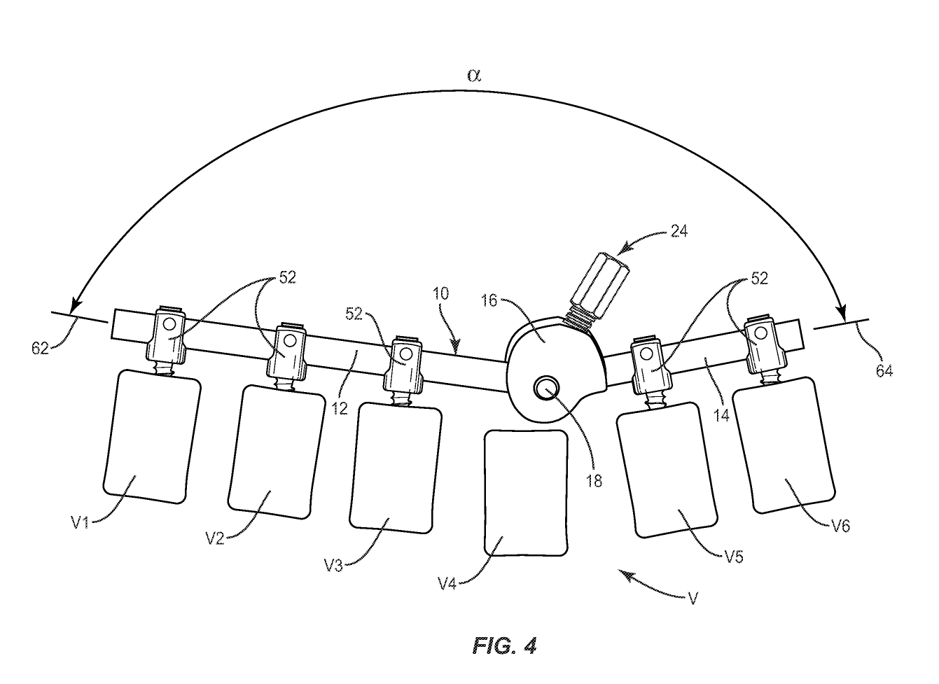

Pivot housing or body 16 includes structural elements or limiters 22 to guide and limit rotation of the second arm 12 relative to the first arm 14. Limiters 22 may be present on opposing sides of the pivot housing 16 to limit an overall angular adjustment range. Limiters 22 are integrally mimed with the pivot housing 16. Pivot housing 16 defines a cavity having, for example, a slot 40 (FIG. 2) that allows second arm 12 to rotate therein. Slot 40 extends such that second arm 12 can rotate through an angle α (see FIG. 4), in the direction shown by arrow “A”. It is contemplated that angle may be in a range of about 10 degrees to about 300 degrees, although greater or lesser limitations may be placed on the range of angles by limiters 22, e.g., about 30 degrees to about 270 degrees.

Pivot housing 16 includes an opening or recess 20 configured to receive a dowel or pin 18. Pin 18 spans the pivot housing 16 and is received and is seated in the openings 20 on opposite sides of the pivot housing 16. The pin 18 permits rotational motion of the second arm 12 relative to the pivot housing and the first arm 14. In the embodiment depicted in FIG. 1, an in-line arrangement is shown where the first arm 14 and the second arm 12 have a longitudinal axis that passes through the pivot point (pin 18). It should be understood that the pivoting housing 16 may he configured to provide an offset between the longitudinal axis of the first arm 14 and the longitudinal axis of the second arm 12.

During operation, the first arm 14 is positioned along a skeletal structure, e.g., a spine. The first arm 14 may be temporarily or permanently affixed to one or more vertebrae. The second arm 12 is angularly adjusted in the direction of arrow “A” to conform to a given configuration of the spinal column. The configuration of provisional spinal rod 10 includes an angular adjustment of the second arm 12 about the pivot point or pin 18. The second arm 12 is then locked in its angularly adjusted place using a locking mechanism 24. The locking mechanism 24 includes a set screw in the illustrative embodiment depicted; however, other locking mechanisms may be employed instead of or in additional to a set screw. Such locking mechanisms may include, for example, clips, a biased engagement arm, a levered cam arrangement or a pin and slot connection.

Referring to FIG. 2, an exploded view of the provisional spinal rod 10 is depicted and provides additional details of an illustrative locking mechanism 24. Second arm 12 includes a cylindrical end portion 30 having an outer surface configured with a three dimensional structure or features 36 having interlocking features, protrusions, splines, a roughened surface and/or a knurled surface.

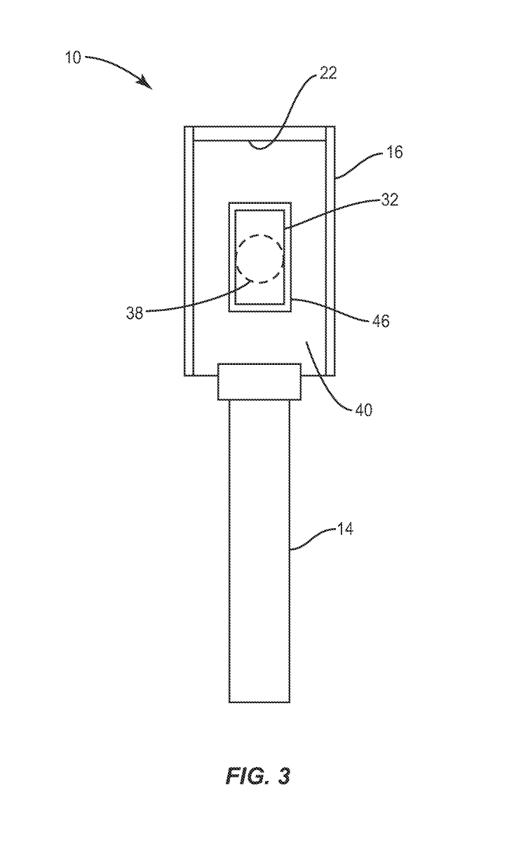

An insert 32 includes an interfacing surface 34 that interfaces with the features 36 of the end portion 30 of the second arm 12. Insert 32 enhances fixation of end portion 30 with a pivot housing 16 to facilitate disposal of provisional spinal rod 10 in a locking configuration. In this illustrative embodiment, the insert 32 is seated within a recess 46 (FIG. 3) within the pivot housing 16 and in contact with an end of threaded portion 38 of a set screw 42.

Pin 18 passes through the second arm 12 to create a pivot joint with the pivot housing 16. Insert 32 may be formed from a rigid or semi-rigid material and include features on a surface 34 that correspond to the features 36 of the end portion 30 of the second arm 12. In one embodiment, insert 32 may include a soil or flexible material that conforms with the features 36 of the end portion 30 of the second arm. 12 when compressed by the locking mechanism 24 (e,g., threaded end portion 38). It is envisioned that insert 32 may include a gripping surface fir engagement with end portion 30, which may include a projection and/or other surface configuration to enhance engagement with end portion 30, such as, for example, splined, ribbed, rough, arcuate, undulating, mesh, porous, semi-porous, dimpled, adhesive and/or textured according to the requirements of a particular application. It is further envisioned that all or only a portion of insert 32 may be fabricated from a flexible, semi-rigid awl/or rigid material, such as those materials described above.

In one embodiment, the second arm 12 is adjustable relative to the first arm 14 such that an angular position is set at an appropriate angle a (FIG, 4), for example, along a curvature of a spine. The appropriate angle is then locked in place by employing the locking mechanism 24 and insert 32, described above. In this embodiment, the locking mechanism 24 includes the set screw 42, which is threaded into the pivot housing 16 to engage the insert 32 and apply pressure thereto. The insert 32 contacts the interlocking features 36 of the second arm 12 to resist any further angular motion. The insert 32 may include corresponding or matching protrusions that are present on the end portion 30 of the second arm 12 similar to interlocking features 36. In other embodiments, the insert 32 may include a flexible pad that occupies a void defined between the interlocking features 36 when pressure is applied by the set screw 42. The set screw 42 preferably permits for hand tightening to employ the locking mechanism 24. In one embodiment, a portion 44 of the set screw 42 is configured as a hex nut, thumb screw or other feature to enable hand or tool tightening.

In assembly, operation and use, the spinal implant system is employed during a surgical procedure, such as, for example, a PSO, VCR or other correction treatment to treat, for example, scoliosis and/or kyphosis of a spine. It is contemplated that one or all of the components of the spinal implant system can be delivered or implanted as a pre-assembled device or can be assembled in-situ. It is further contemplated that one or all of the components of the spinal implant system may be completely or partially revised, removed or replaced.

As shown in FIG. 4, the spinal implant system can be employed with a surgical correction treatment of an applicable condition or injury of an affected section or sections of a spinal column, such as, for example, vertebrae V, which includes a plurality of vertebra V1-V6, and adjacent areas within a body. In use, to treat vertebrae V, a medical practitioner obtains access to a surgical site including vertebra V1-V6 in any appropriate manner, such as through incision and retraction of tissues. It is envisioned that the spinal implant system can be used in any existing surgical method or technique including open surgery, mini-open surgery, minimally invasive surgery and percutaneous surgical implantation, whereby a section of vertebrae V including vertebra V1-V6 are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of the spinal implant system. A preparation instrument (not shown) can be employed to prepare tissue surfaces of vertebrae V, as well as for aspiration and irrigation of a surgical region according to the requirements of a particular surgical application.

Pilot holes are made in selected vertebra of vertebrae V for receiving fixation elements, such as, for example, bone fasteners 52. Each bone fastener 52 is inserted or otherwise engaged with a particular vertebra, according to the particular requirements of the surgical treatment. Bone fasteners 52 each include a receiver defining an implant cavity configured for disposal of arms 14, 12 and/or the spinal rod implant for which the provisional spinal rod provides a template. It is envisioned that the spinal implant system may include one or a plurality of fixation elements corresponding to the number, configuration and procedure.

Provisional spinal rods 10 are employed as provisional and/or working rods to temporarily support vertebrae V during a corrective procedure and/or provide a template configuration for spinal rod implants. It is contemplated that the spinal implant system may include one or a plurality of provisional spinal rods 10. It is further contemplated that the plurality of spinal rods 10 may be disposed in various alternate orientations, such as, for example, side by side, parallel, transverse and/or other angular orientations such as acute or obtuse, co-axial and/or may be offset or staggered. It is envisioned that the plurality of spinal rods 10 may provide a template configuration for spinal rod implants, such as, implantable, final, permanent, removable, non-removable, bio-absorbable, resorbable and/or bio-degradable.

A first provisional spinal rod 10 is attached to vertebrae V. A first arm 14 of the first provisional rod 10 is fixed with a bone fastener 52 to a pedicle of a first vertebra V6. A second arm 12 of the first provisional rod 10 is rotated relative to first arm 14 of the first provisional rod 10 according to an orientation of a second vertebra V1 of vertebrae V relative to the first vertebra V6, as described above. Arms 12, 14 are relatively rotated to an angle α, measured from their respective longitudinal axes, 62, 64, corresponding to the orientation of the second vertebra V1 relative to the first vertebra V6. The pivot point of angle α is about a transverse axis (into the page) along the axis of the pin 18. The first provisional rod 10 is selectively locked with locking mechanism 24 and insert 32, as described above, such that arms 12, 14 are disposed in the orientation in a template configuration for a first spinal rod implant that will be implanted and fixed with vertebra V6, V1 in a similar orientation to the first provisional rod 10. Arm 14 is attached to vertebra V5 via a bone fastener 52. Arm 12 is attached to vertebrae V2 and V3 via bone fasteners 52.

A second provisional spinal rod 10 (not shown) is attached to vertebrae V. A first arm 14 of the second provisional rod 10 is fixed with a bone fastener 52 to a pedicle of a first vertebra V6. A second arm 12 of the second provisional rod 10 is rotated relative to first arm. 14 of the second provisional rod 10 according to the orientation of the second vertebra V1 relative to the first vertebra V6. Arms 12, 14 of the second provisional rod 10 are relatively rotated to angle α corresponding to the orientation of the second vertebra V1 relative to the first vertebra V6. The second provisional rod 10 is selectively locked with locking mechanism 24 and insert 32 such that arms 12, 14 are disposed in the orientation in a template configuration for a second spinal rod implant that will be implanted and fixed with vertebra. V6, V1 in a similar orientation to the second provisional rod 10. Arm 14 of the second provisional rod 10 is attached to vertebra V5 via a bone fastener 52. Arm 12 of the second provisional rod 10 is attached to vertebrae V2 and V3 via bone fasteners 52.

The first and second provisional rods 10 are temporarily fixed with vertebrae V, as described above, to temporarily stabilize and fix vertebrae V in the orientation, according to the requirements of a particular procedure. This configuration facilitates performance of a particular surgical procedure, such as, for example, a PSO or VCR. Upon completion of a surgical procedure, the first provisional rod 10 is removed from vertebrae V adjacent bone fasteners 52. The locking mechanism 24 remains selectively locked such that arms 12, 14 of first provisional rod 10 remain disposed in the orientation of the second vertebra V1 relative to the first vertebra V6, and arms 12, 14 are relatively disposed at angle α. The second provisional rod 10 remains attached with the first and second vertebra V6, V1 to maintain temporary stabilization of vertebrae V.

In the orientation of the second vertebra V1 relative to the first vertebra V6, first provisional rod 10 provides a template configuration for a final spinal rod implant. The first provisional rod 10 provides a configuration and/or measuring device such that a final spinal rod implant can be formed by bending. As such, a first spinal rod implant (not shown) is formed, delivered and reduced with the receivers of the previously implanted bone fasteners 52 corresponding to first provisional rod 10.

The second provisional rod 10 is removed from vertebrae V adjacent bone fasteners 52. The locking mechanism 24 of the second provisional rod 10 remains selectively locked such that arms 12, 14 of second provisional rod 10 remain disposed in the orientation of the second vertebra V1 relative to the first vertebra V6, and arms 12, 14 are relatively disposed at angle α. The first spinal rod implant remains attached with the first and second vertebra V6, V1 to stabilize vertebrae V.

In the orientation of the second vertebra V1 relative to the first vertebra V6, second provisional rod. 10 provides a template configuration for a final spinal rod implant. The second provisional rod 10 provides a configuration and/or measuring device such that a final spinal rod implant can be formed by bending. As such, a second spinal rod implant (not shown) is formed, delivered and reduced with the receivers of the previously implanted bone fasteners 52 corresponding to second provisional rod 10. The first and second spinal rod implants remain attached with the first and second vertebra V6, V1 to stabilize vertebrae V. Upon completion of a procedure, the surgical instruments and assemblies are removed and the incision is closed.

It is contemplated that arms 12, 14 can be secured at an appropriate angle to follow the curvature of a spine or to hold a desired position of a spine. The spinal implant system may be configured for disposal along a plurality of vertebral levels.

The spinal implant system can be used with various bone screws, mono-axial screws, pedicle screws or multi-axial screws used in spinal surgery. In one embodiment, the spinal implant system includes an agent, which may be disposed, packed or layered within, on or about the components and/or surfaces of bone fasteners 52. It is envisioned that the agent may include bone growth promoting material, such as, for example, bone graft to enhance fixation of bone fasteners 52 with vertebrae V.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (EDF) and cytokines. The components of the spinal implant system can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may he employed to access, view and repair spinal deterioration or damage, with the aid of the spinal implant system.

It is contemplated that the components of the spinal implant system may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. It is further contemplated that the components of the spinal implant system and method of use may be used to prevent or minimize curve progression in individuals of various ages.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A provisional spinal rod comprising:
a body;
a first arm extending between a first end and a second end connected to the body, the first arm being configured for attachment to at least a first vertebra;
a second arm extending between a first end and a second end connected to the body, the second arm being configured for attachment to at least a second vertebra disposed in an orientation relative to the first vertebra, the second arm being rotatable about the body and relative to the first arm;
a locking mechanism coupled to the body; and
an insert disposed between the locking mechanism and the second arm,
wherein the arms are relatively rotatable and selectively lockable with the locking mechanism and the insert in the orientation in a template configuration for an implant rod.

2. A provisional spinal rod as recited in claim 1, wherein the body forms a cavity having a slot for receiving the second arm.

3. A provisional spinal rod as recited in claim 1, wherein the body includes at least one limiter to limit an angular range of the second arm.

4. A provisional spinal rod as recited in claim 1, wherein the body is configured to receive a pin to provide an axis of rotation for the second arm.

5. A provisional spinal rod as recited in claim 1, wherein the second arm includes a surface configured to interface with the insert such that the insert engages the surface to lock the orientation of the second arm.

6. A provisional spinal rod as recited in claim 5, wherein the surface includes a three dimensional structure to interface with the insert.

7. A provisional spinal rod as recited in claim 6, wherein the three dimensional structure includes at least one of splines, protrusions or teeth.

8. A provisional spinal rod as recited in claim 1, wherein the body forms a recess for receiving the insert.

9. A provisional spinal rod as recited in claim 8, wherein the insert is configured to engage a surface of the second arm to resist motion from the orientation.

10. A provisional spinal rod as recited in claim 8, wherein the locking mechanism includes a set screw threaded into the body and configured to apply pressure to the insert to lock the orientation of the second arm.

11. A provisional spinal rod as recited in claim 1, wherein the insert includes a compressible material that conforms to a surface of the second arm.

12. A provisional spinal rod as recited in claim 1, wherein the orientation includes an angle defined between longitudinal axes of the second arm and the first arm.

13. A provisional spinal rod as recited in claim 1, wherein the first arm and the second arm are configured for attachment to at least one fixation element.

14. A provisional spinal rod as recited in claim 13, wherein the at least one fixation element is configured for connecting to a vertebra.

15. A provisional spinal rod as recited in claim 1, wherein a longitudinal axis of the first arm is coincident with a pivot point of the second arm.

16. A provisional spinal rod as recited in claim 1, wherein the body is configured to provide an offset between a pivot point of the second arm and a longitudinal axis of the first arm.

17. A provisional spinal rod as recited in claim 1, wherein second arm includes an end portion configured to receive a pin and the body includes structures for receiving the pin to create a pivot point for the second arm.

18. A provisional spinal rod as recited in claim 1, wherein the locking mechanism is hand activated.

19. A spinal implant system comprising:
a provisional spinal rod comprising:
a body forming a cavity having a slot and forming a recess, the body being configured to receive a pin,
a first arm rigidly connected to the body and being configured for attachment to a first vertebra,
a second arm having an end portion configured to receive the pin therethrough to form a pivot connection with the body for rotation of the second arm relative to the first arm, the second arm including a surface having a three dimensional structure, the second arm being configured for attachment to a second vertebra disposed in an orientation relative to the first vertebra,
a locking mechanism coupled to the body and including a set screw threaded into the body, and
an insert disposed in the recess of the body, the insert providing an interface between the set screw and the surface of the second arm, the insert being configured to interface with the three dimensional structure such that the set screw applies pressure to the insert against the three dimensional structure to lock the orientation of the second arm relative to the first arm, wherein the arms are relatively rotatable and selectively lockable with the locking mechanism in the orientation in a template configuration; and
a spinal rod implant having a configuration based on the template configuration of the provisional spinal rod.

20. A method for treating a spinal disorder, the method comprising the steps of:
providing access to a surgical site adjacent a selected section of a spine;
providing at least one provisional spinal rod including a body, a first arm extending between a first end and a second end connected to the body, a second arm extending between a first end and a second end connected to the body, the second arm being rotatable about the body and relative to the first arm, a locking mechanism coupled to the body, and an insert disposed between the locking mechanism and the second arm;
connecting the first arm with a first vertebra.;
connecting the second arm with a second vertebra, disposed in an orientation relative to the first vertebra;
relatively rotating the arms and selectively locking the locking mechanism with the insert such that the arms are disposed in the orientation in a template configuration; and
implanting at least one spinal rod implant with the first vertebra and the second vertebra, the at least one spinal rod implant having a configuration based on the template configuration of the at least one provisional spinal rod.
